(19) United States
(12) Patent Application Publication
(10) Pub. No.: US 2026/0272902 A1
(43) Pub. Date: Sep. 17, 2026

(54) CRYSTALLINE ADIPIC ACID SALT FORM OF A CCR6 ANTAGONIST

(71) Applicant: Idorsia Pharmaceuticals Ltd, Allschwil (CH)

(72) Inventors: Tony FLEISCHER, Allschwil (CH); Daniel MARCHAL, Allschwil (CH); Timo RAGER, Allschwil (CH); Gabriel SCHAEFER, Allschwil (CH); Cécilia ZOUGGARI, Allschwil (CH)

(21) Appl. No.: 19/136,271

(22) PCT Filed: Dec. 5, 2023
(86) PCT No.: PCT/EP2023/084328

§ 371 Date: Jun. 5, 2025

(30) Foreign Application Priority Data
Dec. 6, 2022 (WO) PCT/EP2022/084559

(51) Int. Cl. A61K 31/4439; A61K 9/48; C07D 413/14

(52) U.S. Cl. CPC A61K 31/4439; A61K 9/4825; A61K 9/485; A61K 9/4858; A61K 9/4866; C07D 413/14

(57) ABSTRACT

The present invention relates to a crystalline form of 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol adipic acid salt and its use in the treatment or prevention of various diseases or disorders such as cancer or inflammatory/autoimmune diseases or disorders.

The present invention relates to a novel crystalline form of 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol adipic acid salt, a process for the preparation thereof, pharmaceutical compositions comprising the crystalline form, and its use in the treatment or prevention of various diseases or disorders ameliorated by modulating chemokine receptor 6 (CCR6). Such diseases or disorders include cancer and inflammatory/autoimmune diseases or disorders such as psoriasis.

The synthesis of 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol represented by Formula (I) (hereinafter also referred to as “COMPOUND”), its biological activity as CCR6 antagonist and its medical use are disclosed in WO2021219849.

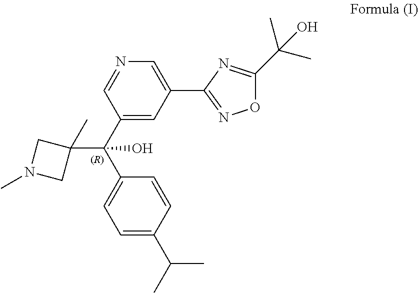

Despite substantial experimentation, a crystalline form of COMPOUND or a crystalline salt form of COMPOUND, suitable as active pharmaceutical ingredient (API) proved difficult to find. In accordance with the present invention a novel crystalline form of 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol adipic acid salt (hereinafter also referred to as “COMPOUND adipic acid salt”) has been found, which in view of the potential use of COMPOUND as API may have advantageous properties. Such properties may include improved aqueous solubility, better pharmacological and/or pharmacokinetic properties (e.g. bioavailability), lower hygroscopicity, improved chemical and/or physical (e.g. thermal) stability, better reproducibility in manufacturing, better defined particle size and/or morphology, and/or better bulk properties such as density or flowability.

### DESCRIPTION OF THE FIGURES

FIG. 1 shows the X-ray powder diffractogram of COMPOUND adipic acid salt in crystalline form A as obtained in Examples 1a to 1d. The X-ray powder diffractogram as measured by the method disclosed herein shows peaks having a relative intensity, as compared to the most intense peak in the diagram, of the following percentages (relative peak intensities given in parenthesis) at the indicated angles of refraction 2theta (peaks from the range 5-40° 2theta with relative intensity larger than 7% are reported): 6.9° (24%), 12.2° (7%), 15.0° (9%), 16.4° (8%), 17.8° (15%), 19.2° (68%), 20.3° (100%), 20.9° (8%), 21.5° (12%), 22.6° (44%), 24.7° (9%), 26.8° (16%), 27.7° (10%), 30.3° (8%), and 35.3° (9%). It is understood that, in contrast to the above peak list, only a selection of characteristic peaks is required to characterize the COMPOUND adipic acid salt in crystalline form A fully and unambiguously. It is also understood that relative peak intensities may vary as a result of inter alia preferential orientation or particle size. In the X-ray powder diffractogram of FIG. 1 the angle of refraction 2theta (20) is plotted on the horizontal axis and the counts on the vertical axis.

FIG. 2 shows the thermogravimetric analysis (TGA) of COMPOUND adipic acid salt in crystalline form A, where the relative mass (% of the total mass of the sample) on the vertical axis is displayed against temperature (C).

FIG. 3 shows the differential scanning calorimetry (DSC) analysis of COMPOUND adipic acid salt in crystalline form A, where the heat flow (mW) on the vertical axis is displayed against temperature (° C.).

FIG. 4 shows the gravimetric vapor sorption analysis (GVS) of COMPOUND adipic acid salt in crystalline form A, where the relative change in mass (%) on the vertical axis is displayed against relative humidity (%).

FIG. 5 shows the X-ray powder diffractogram of COMPOUND adipic acid salt in crystalline form B as obtained in Reference Example 1. The X-ray powder diffractogram as measured by the method disclosed herein shows peaks having a relative intensity, as compared to the most intense peak in the diagram, of the following percentages (relative peak intensities given in parenthesis) at the indicated angles of refraction 2theta (peaks from the range 5-40° 2theta with relative intensity larger than or equal to 19% are reported): 6.2° (63%), 7.0° (45%), 8.8° (27%), 12.4° (23%), 12.9° (34%), 14.8° (30%), 15.8° (29%), 16.7° (30%), 18.3° (47%), 19.6° (100%), 21.3° (30%), 27.6° (19%), 29.5° (19%). It is understood that, in contrast to the above peak list, only a selection of characteristic peaks is required to characterize the COMPOUND adipic acid salt in crystalline form B fully and unambiguously. In the X-ray powder diffractogram of FIG. 5 the angle of refraction 2theta (20) is plotted on the horizontal axis and the counts on the vertical axis.

FIG. 6 shows the thermogravimetric analysis (TGA) of COMPOUND adipic acid salt in crystalline form B, where the relative mass (% of the total mass of the sample) on the vertical axis is displayed against temperature (C).

FIG. 7 shows the differential scanning calorimetry (DSC) analysis of COMPOUND adipic acid salt in crystalline form B, where the heat flow (mW) on the vertical axis is displayed against temperature (° C.).

### DETAILED DESCRIPTION OF THE INVENTION

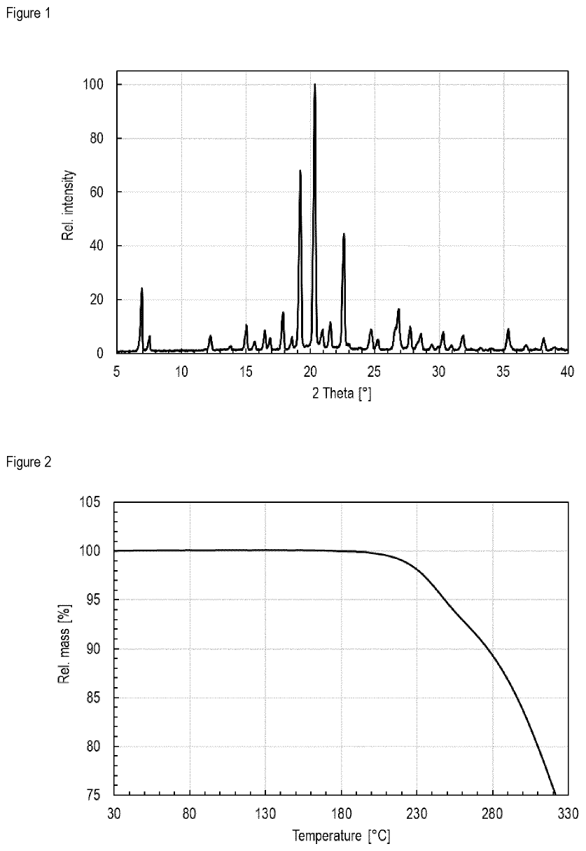

1) One aspect of the present invention relates to a crystalline form of the COMPOUND 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol adipic acid salt, wherein the crystalline form is characterized by the presence of peaks in the X-ray powder diffractogram at the following angles of refraction 20:6.9°, 17.8° and 19.2°.
2) One embodiment relates to a crystalline form of the COMPOUND adipic acid salt according to embodiment 1), wherein the crystalline form is characterized by the presence of peaks in the X-ray powder diffractogram at the following angles of refraction 20:6.9°, 12.2°, 16.8°, 17.8° and 19.2°.
3) Another embodiment relates to a crystalline form of the COMPOUND adipic acid salt according to embodiment 1), wherein the crystalline form is characterized by the presence of peaks in the X-ray powder diffractogram at the following angles of refraction 20:6.9°, 12.2°, 15.6°, 16.4°, 16.8°, 17.8°, 19.2°, 20.3°, 21.5°, and 22.6°.
4) Another embodiment relates to a crystalline form of the COMPOUND adipic acid salt according to any one of embodiments 1) to 3), wherein the crystalline form essentially shows the X-ray powder diffraction pattern as depicted in FIG. 1.
5) Another embodiment relates to a crystalline form of the COMPOUND adipic acid salt, especially according to any one of embodiments 1) to 4), wherein the crystalline form is characterized by the presence of an endothermic peak in the differential scanning calorimetry (DSC) thermogram at 163.5±5° C. (especially 163.5±2° C.) [as determined by the DSC method described herein].

A salt of adipic acid may be referred to as adipate. The terms “COMPOUND adipic acid salt” or “2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol adipic acid salt” may thus be referred to as “COMPOUND adipate” or “2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol adipate”, respectively. It is understood that the COMPOUND adipic acid salt in crystalline form A comprises from 0.9 to 1.1 (notably from 0.95 to 1.05; especially 1) equivalent of adipic acid and may be referred to as COMPOUND mono adipate in crystalline form A or COMPOUND adipic acid salt (1:1) in crystalline form A.

Where the plural form is used for compounds, solids, forms, hydrates, compositions, diseases and the like, this is intended to also mean a single compound, solid, form, hydrate, composition, disease or the like.

It is understood that the crystalline salt of the present invention may comprise non-coordinated solvent (e.g. water) and/or coordinated solvent (e.g. water). Non-coordinated solvent is used herein as a term for physisorbed or physically entrapped solvent. (Definitions according to Polymorphism in the Pharmaceutical Industry (Ed. R. Hilfiker, VCH, 2006), Chapter 8: U. J. Griesser: The Importance of Solvates). The crystalline salt of the present invention may especially comprise essentially no coordinated solvent (e.g. water).

For avoidance of any doubt, whenever one of the above embodiments refers to “peaks in the X-ray powder diffractogram at the following angles of refraction 2θ”, said X-ray powder diffractogram is obtained by using combined Cu Kα1 and Kα2 radiation, without Kα2 stripping; and it should be understood that the accuracy of the 2θ values as provided herein is in the range of +/−0.1-0.2°. Notably, when specifying an angle of refraction 2theta (2θ) for a peak in the invention embodiments and the claims, the 2θ value given is to be understood as an interval from said value minus 0.2° to said value plus 0.2° (2θ+/−0.2°); and preferably from said value minus 0.1° to said value plus 0.1° (2θ+/−0.1°).

Definitions provided herein are intended to apply uniformly to the subject matter as defined in any one of the embodiments disclosed herein, and, mutatis mutandis, throughout the description and the claims unless an otherwise expressly set out definition provides a broader or narrower definition. It is well understood that a definition or preferred definition of a term or expression defines and may replace the respective term or expression independently of (and in combination with) any definition or preferred definition of any or all other terms or expressions as defined herein.

When defining the presence of peaks in e.g. an X-ray powder diffractogram, a common approach is to do this in terms of the S/N ratio (S=signal, N=noise). According to this definition, when stating that a peak has to be present in an X-ray powder diffractogram, it is understood that the peak in the X-ray powder diffractogram is defined by having an S/N ratio (S=signal, N=noise) of greater than x (x being a numerical value greater than 1), usually greater than 2, especially greater than 3.

In the context with stating that the crystalline form essentially shows an X-ray powder diffraction pattern as depicted in a Figure, respectively, the term “essentially” means that at least the major peaks of the diagram depicted in said figures, i.e. those having a relative intensity of more than 20%, especially more than 10%, as compared to the most intense peak in the diagram, have to be present. However, the person skilled in the art of X-ray powder diffraction will recognize that relative intensities in X-ray powder diffractograms may be subject to strong variations e.g. due to preferred orientation effects that may result in missing peaks or intensity variations of single peaks.

Unless used regarding temperatures, the term “about” placed before a numerical value “X” refers in the current application to an interval extending from X minus 10% of X to X plus 10% of X, and preferably to an interval extending from X minus 5% of X to X plus 5% of X; most preferred is X. In the particular case of temperatures, the term “about” placed before a temperature “Y” refers in the current application to an interval extending from the temperature Y minus 10° C. to Y plus 10° C., preferably to an interval extending from Y minus 5° C. to Y plus 5° C.

Room temperature means a temperature of about 25° C.

Whenever terms such as “between X and Y”; “X to Y”; “from X to Y”; or “X-Y”, are used to describe a numerical range, it is to be understood that the end points “X” and “Y” of the indicated range are explicitly included in the range. For example, if a temperature range is described to be between 40° C. and 80° C. (or 40° C. to 80° C.), this means that the end points 40° C. and 80° C. are included in the range.
6) Another aspect relates to a crystalline form, such as an essentially pure crystalline form, of the COMPOUND adipic acid salt according to any one of embodiments 1) to 5), wherein the crystalline form, such as the essentially pure crystalline form, is obtainable by a process comprising the following steps:
   a) providing a first solution/suspension of about 1 eq. adipic acid in (notably from about 4 to about 10 vol.; especially about 7 vol.) acetone;
   b) providing a second solution comprising about 1 eq. of COMPOUND in (notably from about 4 to about 10 vol.; especially about 7 vol.) acetone;
   c) mixing of the first solution/suspension and the second solution under heating (notably at about 25° C. to about 56° C.; especially at about 40° C. to about 50° C.);
   d) cooling the resulting mixture (notably to a temperature below about 25° C.; especially to about 5 to about 10° C.);
   e) optionally stirring the resulting mixture (notably for at least about 30 min);
   f) isolating the solid residue through a solid-liquid separation; and
   g) drying the solid residue (notably under vacuum).
7) One embodiment relates to a crystalline form, such as an essentially pure crystalline form, of the COMPOUND adipic acid salt according to embodiment 6), wherein the mixing of the first solution/suspension and the second solution is performed by adding the second solution to the first solution/suspension, under heating.

For avoidance of doubt, in embodiment 7) the mixing of the first solution/suspension and the second solution is performed by adding the second solution to the first solution/suspension; and not the first solution/suspension to the second solution.

It is understood that the step of isolating the solid residue through a solid-liquid separation refers to separating the solid phase of a suspension from its liquid phase. It is understood that said isolation may be performed by any method for solid-liquid separation such as filtration (e.g. gravity filtration or vacuum filtration).

After isolation, the solid residue is optionally washed with a solvent such as acetone.
8) Another aspect relates to a process for manufacturing a crystalline form, such as an essentially pure crystalline form, of the COMPOUND adipic acid salt according to any one of embodiments 1) to 7), wherein the process comprises the steps as defined in any one of embodiments 6) or 7).
9) Another aspect relates to a pharmaceutical composition comprising a crystalline form of the COMPOUND adipic acid salt according to any one of embodiments 1) to 7), wherein the pharmaceutical composition further comprises at least one pharmaceutically acceptable carrier material; especially such carrier material is selected from a group comprising alpha lactose monohydrate, beta lactose, mannitol, starch, microcrystalline cellulose, hydroxypropylmethylcellulose, sodium carboxymethylcellulose, sodium starch glycolate, croscarmellose sodium, magnesium stearate, compritol, aerosil, talcum, sodium dodecyl sulfate, ascorbic acid, sodium bicarbonate, calcium hydrogen phosphate, or a mixture thereof.

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the crystalline form of the present invention, optionally in combination with other therapeutically valuable substances, into a galenical administration form together with suitable, non-toxic, inert, pharmaceutically acceptable solid or liquid carrier materials and, if desired, usual pharmaceutical adjuvants.

The crystalline form of the present invention may be used as a medicament, e.g. in the form of a pharmaceutical composition for enteral or parenteral, notably oral administration e.g. in the form of a solid pharmaceutical composition such as capsule or tablet.

The crystalline form of the present invention may be used as a single crystalline form or as a mixture with other crystalline forms and/or amorphous COMPOUND adipic acid salt.
10) Another aspect relates to a crystalline form of the COMPOUND adipic acid salt according to any one of embodiments 1) to 7), for use as a medicament.
11) One embodiment relates to a crystalline form according to any one of embodiments 1) to 7), for use in the prevention or treatment of a disease or disorder, which is mediated (at least in part) by chemokine receptor 6 (CCR6).
12) Another embodiment relates to a crystalline form according to any one of embodiments 1) to 7), for use in the prevention or treatment of cancer; or an inflammatory/autoimmune disease or disorder.

For avoidance of any doubt, if the crystalline form of the present invention is described as useful for the prevention or treatment of certain diseases or disorders, such crystalline form is likewise suitable for use in the preparation of a medicament for the prevention or treatment of said diseases or disorders.
13) Another aspect relates to a method for the prevention or treatment of cancer; or an inflammatory/autoimmune disease or disorder, said method comprising administering to a subject in need of such prevention or treatment an effective amount of the crystalline form according to any one of embodiments 1) to 7).

The term “inflammatory/autoimmune disease or disorder”, as used herein, refers to a disease or disorder selected from a group comprising rheumatoid arthritis; ankylosing spondylitis; spondyloarthritis; psoriasis; psoriatic arthritis; inflammatory skin disorders such as rosacea and hidradenitis suppurativa; Crohn's disease; ulcerative colitis; inflammatory bowel disease; irritable bowel disease; dry eye disease; multiple sclerosis; systemic lupus erythematosus; Sjögren's disease; autoimmune hepatitis; primary sclerosing cholangitis (primary sclerotic cirrhosis); primary biliary cholangitis (primary biliary cirrhosis); posterior uveitis; allergic conjunctivitis; allergic disease in the gastrointestinal tract; endometriosis; diseases of the ocular surface in which elevated levels of IL-17A have been recorded such as meibomian gland dysfunction; graft-versus host disease (GvHD); autoimmune keratitis; filamentary keratitis; dry eye syndrome with rheumatic arthritis; dry eye syndrome without systemic disease; Stevens-Johnson syndrome; psoriasis including plaque psoriasis, guttate psoriasis, inverse psoriasis, pustular psoriasis, and erythrodermic psoriasis; autoimmune keratitis; autoimmune bullous diseases; filamentary keratitis; Pityriasis rubra pilaris; autoimmune uveitis; allergic conjunctivitis; asthma; allergic disease of the gastrointestinal tract; Type 1 diabetes; meibomian gland dysfunction; juvenile arthritis; juvenile rheumatoid arthritis; systemic onset rheumatoid arthritis; pauciarticular rheumatoid arthritis; pauciarticular juvenile rheumatoid arthritis; polyarticular rheumatoid arthritis; enteropathic arthritis; juvenile Reiter's syndrome; juvenile ankylosing spondylitis; seronegative enthesopathy and arthropathy (SEA) syndrome; reactive arthritis, reactive arthropathy; psoriatic arthropathy; juvenile enteropathic arthritis; polymyalgia rheumatica; enteropathic spondylitis; juvenile idiopathic arthritis; juvenile psoriatic arthritis; juvenile rheumatoid arthritis; acute pancreatitis; chronic pancreatitis; giant cell arteritis; and secondary osteoarthritis from inflammatory diseases.

The term “inflammatory/autoimmune disease or disorder”, as used herein, especially refers to a disease or disorder selected from a group comprising rheumatoid arthritis; ankylosing spondylitis; spondyloarthritis; psoriasis; psoriatic arthritis; inflammatory skin disorders e.g. rosacea; Crohn's disease; ulcerative colitis; irritable bowel disease; inflammatory bowel disease; dry eye disease; multiple sclerosis; systemic lupus erythematosus; hidradenitis suppurativa; Sjögren's disease; autoimmune hepatitis; primary sclerosing cholangitis; primary biliary cholangitis; psoriasis including plaque psoriasis, guttate psoriasis, inverse psoriasis, pustular psoriasis, erythrodermic psoriasis; autoimmune keratitis; filamentary keratitis; Pityriasis rubra pilaris; autoimmune bullous diseases; autoimmune uveitis; allergic conjunctivitis; asthma; allergic disease of the gastrointestinal tract; type 1 diabetes; endometriosis; meibomian gland dysfunction; and graft-versus host disease.

The term “inflammatory/autoimmune disease or disorder”, as used herein, in particularly refers to a disease or disorder selected from a group comprising psoriasis; psoriatic arthritis; rheumatoid arthritis; ankylosing spondylitis; spondyloarthritis; inflammatory skin disorders e.g. rosacea; autoimmune bullous diseases; hidradenitis suppurativa; Crohn's disease; ulcerative colitis; irritable bowel disease; dry eye disease; multiple sclerosis; systemic lupus erythematosus; Sjögren's disease; autoimmune hepatitis; and primary sclerosing cholangitis.

The term “inflammatory/autoimmune disease or disorder”, as used herein, more particularly refers to a disease or disorder selected from a group comprising psoriasis (preferred) including plaque psoriasis, guttate psoriasis, inverse psoriasis, pustular psoriasis, erythrodermic psoriasis; psoriatic arthritis; hidradenitis suppurativa; ankylosing spondylitis; and primary sclerosing cholangitis.

The term “cancer”, as used herein, refers to a cancer selected from a group comprising skin cancer e.g. melanoma (superficial spreading, nodular, lentigo maligna and acral lentiginous melanoma); advanced melanoma; metastatic melanoma; Merkel cell carcinoma; Kaposi sarcoma; basal cell carcinoma; squamous cell carcinoma; and pre-cancerous skin lesions such as actinic keratosis; lung cancer including small cell lung cancer and non-small (SCLC, NSCLC) such as squamous and non-squamous NSCLC; pleuropulmonary blastoma and tracheobronchial tumors; bladder cancer including urinary bladder cancer; urothelial cell carcinoma; mesothelioma; renal carcinomas including renal cell carcinoma (RCC) such as clear cell RCC; papillary RCC; chromophobe RCC; non-clear cell RCC; unclassified RCC; metastatic renal cell carcinoma; metastatic renal clear cell carcinoma; renal parenchymal carcinoma; gastro-intestinal cancers including colorectal cancer; metastatic colorectal cancer; familial adenomatous polyposis (FAP); rectum carcinoma; colon carcinoma; colorectal adenoma; colorectal adenocarcinoma; colorectal cancer liver metastases; hereditary non-polyposis colorectal cancer; esophageal cancer; gastric cancer; advanced gastric cancer; gallbladder cancer; cholangiocarcinoma; hepatocellular carcinoma; pancreatic cancer such as pancreatic adenocarcinoma or pancreatic ductal (adeno) carcinoma; pancreatic neuroendocrine tumors; endometrial cancer; ovarian cancer; prostate cancer including castrate-resistant prostate cancer; brain tumors including brain metastases, malignant gliomas, glioblastoma multiforme, medulloblastoma, meningiomas, astrocytoma; peripheral neuroectodermal tumors; oligoastrocytic tumors; oligodendrogliomas; ependymal tumors; anaplastic astrocytoma; pilocytic astrocytoma; craniopharyngioma; spinal cord tumors; brain stem glioma; central nervous system atypical teratoid/rhabdoid tumor; medulloblastoma; central nervous system germ cell tumors; craniopharyngioma; ependymoma; neuroblastoma; head and neck cancer such as esthesioneuroblastoma; cervical cancer; advanced cervical cancer; breast cancer including normal-like, basal-like, claudin-low, HER2 positive, luminal-A, luminal-B and triple negative breast carcinoma; pregnancy breast cancer and male breast cancer; oral tumors; nasopharyngeal tumors; heart tumors; thoracic cancer; lymphomas such as Hodgkin lymphoma, non-Hodgkin lymphoma, Burkitt lymphoma; primary intra-ocular B-Cell lymphoma; diffuse large B-cell lymphoma; primary mediastinal large B-cell lymphoma; mucosa-associated lymphoid tissue (MALT) lymphoma; gastric MALT lymphoma; cutaneous T-cell lymphoma; primary central nervous system lymphoma; Sezary syndrome and Waldenström macroglobulinemia; leukemia such as acute lymphoblastic leukemia; acute myeloid leukemia; chronic lymphocytic leukemia; chronic myelogenous leukemia; hairy cell leukemia; chronic myeloid leukemia; adult T-cell leukemia; carcinomas; adenocarcinomas; thyroid carcinoma including papillary thyroid carcinoma and medullary thyroid carcinoma choriocarcinoma; sarcomas including Ewing's sarcoma; bone cancer such as osteosarcoma; high-grade osteosarcoma; rhabdomyosarcoma; Ewing sarcoma; malignant fibrous histiocytoma of the bone; chordoma; soft tissue sarcoma; myeloma; multiple myelomas; labial carcinoma; larynx carcinoma; hypopharynx carcinoma; tongue carcinoma; salivary gland carcinoma; cervix carcinoma; uterine corpus carcinoma; endometrium carcinoma; chorion carcinoma; testis carcinoma; urinary carcinoma; bronchial carcinoma; basalioma; teratoma; retinoblastoma; choroid melanoma; seminoma; chondrosarcoma; myosarcoma; liposarcoma; fibrosarcoma; plasmacytoma; hepatocarcinoma; advanced liver cancer; gastrointestinal stromal tumors; neuroendocrine tumors; bile duct cancer; appendix cancer; gastrointestinal carcinoid tumor; carcinoid tumor; islet cell tumor; small intestine cancer; stomach cancer; adrenocortical carcinoma; parathyroid cancer; paraganglioma; pheochromocytoma; pituitary tumor; penile cancer; renal pelvis and ureter cancer; testicular cancer; urethral cancer; Wilms tumor; extracranial germ cell tumor; extragonadal germ cell tumor; fallopian tube cancer; gestational trophoblastic tumor; primary peritoneal cancer; vaginal cancer; vulvar cancer; hypopharyngeal cancer; laryngeal cancer; papillomatosis cancer; lip and oral cavity cancer; metastatic squamous neck cancer; mouth cancer; nasopharyngeal cancer; oropharyngeal cancer; paranasal sinus and nasal cavity and paranasal sinus cancer; parathyroid cancer; pharyngeal cancer; throat cancer; chronic myeloproliferative neoplasm; Langerhans cell histiocytosis; plasma cell neoplasm; myelodysplastic syndromes; myeloproliferative neoplasm; midline tract carcinoma; virally induced tumors; and diseases involving CCR6 and/or CCL20 mediated metastasis, chemotaxis, cell adhesion, trans-endothelial migration, cell proliferation and/or survival.

The term “cancer”, as used herein, especially refers to a cancer selected from a group comprising lymphoma including T cell lymphoma and primary mediastinal B-cell lymphoma; brain cancer including glioma and glioblastoma; breast cancer including triple negative breast cancer; colorectal cancer; hepatocarcinoma; renal cell carcinoma; lung cancer including non-small cell lung cancer and small cell lung cancer; gastric cancer; melanoma including Merkel cell carcinoma, cutaneous squamous cell carcinoma and malignant melanoma; bladder cancer; head and neck cancer including squamous cell head and neck carcinoma; Hodgkin's lymphoma; cervical cancer; endometrial cancer; colon cancer; gastrointestinal stromal tumors; pancreatic cancer; prostatic cancer; leukemia including acute myeloid leukemia; ovarian cancer; oesophageal carcinomas; mesothelioma; neuroblastoma; sarcoma e.g. high-grade osteosarcoma; astrocytoma; myeloma; urothelial cancer including locally advanced and metastatic urothelial cancer; MSI-H or dMMR cancer; rectal cancer; laryngeal cancer; salivary adenocarcinoma; multiple myeloma; cholangiocarcinoma; oral squamous cell carcinoma; thyroid cancer; and esophagogastric junction cancer.

The term “cancer”, as used herein, in particularly refers to a cancer selected from a group comprising lymphoma, (e.g. T cell lymphoma); brain cancer (e.g. glioma or glioblastoma); breast cancer; colorectal cancer; hepatocarcinomas; renal cell carcinoma; lung cancer; and gastric cancer.

The COMPOUND 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol, especially the COMPOUND adipic acid salt in crystalline form A, when used for the prevention or treatment of a cancer, such use includes the use as a single therapeutic agent and the use in combination with one or more chemotherapy agents and/or radiotherapy and/or targeted therapy (notably in combination with targeted therapy; especially in combination with immune checkpoint inhibitors such as those targeting the programmed cell death receptor 1 (PD-1 receptor) or its ligand PD-L1 (Feig C et al, PNAS 2013).

The terms “radiotherapy” or “radiation therapy” or “radiation oncology”, refer to the medical use of ionizing radiation in the prevention (adjuvant therapy) and/or treatment of cancer, including external and internal radiotherapy.

The term “targeted therapy” refers to the prevention/prophylaxis (adjuvant therapy) and/or treatment of cancer with one or more anti-neoplastic agents such as small molecules or antibodies which act on specific types of cancer cells or stromal cells. Some targeted therapies block the action of certain enzymes, proteins, or other molecules involved in the growth and spread of cancer cells. Other types of targeted therapies help the immune system kill cancer cells (immunotherapies); or deliver toxic substances directly to cancer cells and kill them. An example of a targeted therapy which is in particular suitable to be combined with the COMPOUND 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol, especially the COMPOUND adipic acid salt in crystalline form A, is immunotherapy, especially immunotherapy targeting the programmed death 1 (PD-1) receptor or its ligand PD-L1 (Feig C et al, PNAS 2013).

Immunotherapy further refers to (i) an agonist of a stimulatory (including a co-stimulatory) receptor or (ii) an antagonist of an inhibitory (including a co-inhibitory) signal on T cells, both of which result in amplifying antigen-specific T cell responses (often referred to as immune checkpoint regulators). Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-HI (PD-LI), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-IBBL, CD137 (4-IBB), TRAIL/Apo2-L, TRAILR1/DR4, TRAILR2/DR5, TRAILR3, TRAILR4, OPG, RANK, RANKL, TWEAKR/Fnl4, TWEAK, BAFFR, EDAR, XEDAR, TACI, APRIL, BCMA, LTpR, LIGHT, DcR3, HVEM, VEGi/TLIA, TRAMP/DR3, EDAR, EDAI, XEDAR, EDA2, TNFRI, Lymphotoxin a/TNFp, TNFR2, TNFa, LTPR, Lymphotoxin a 1p2, FAS, FASL, RELT, DR6, TROY, NGFR.

When used in combination with the COMPOUND 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol, especially the COMPOUND adipic acid salt in crystalline form A, the term “targeted therapy” especially refers to agents such as:
a) Epidermal growth factor receptor (EGFR) inhibitors or blocking antibodies (such as Gefitinib, Erlotinib, Afatinib, Icotinib, Lapatinib, Panitumumab, Neratinib, Osimertinib, Dacomitinib, Almonertinib, Tucatinib, Zalutumumab, Nimotuzumab, Matuzumab, Cetuximab), as well as trastuzumab (HERCEPTIN);
b) RAS/RAF/MEK pathway inhibitors (such as Vemurafenib, Sorafenib, Dabrafenib, GDC-0879, PLX-4720, Encorafenib (LGX818), RG7304, Trametinib (GSK1120212), Cobimetinib (GDC-0973/XL518), Binimetinib (MEK 162, ARRY-162), Selumetinib (AZD6244), Sotorasib, Adagrasib, as well as various mutant KRAS-specific, pan RAS, SOS1, and SHP2 inhibitors (Punekar et al., Nature Rev Clin Oncology, 19 (10): 637-655));
c) Janus kinase (JAK) inhibitors (such as Ruxolitinib, Itacitinib, Momelotinib, Fedratinib);
d) Aromatase inhibitors (such as Exemestane, Letrozole, Anastrozole, Vorozole, Formestane, Fadrozole);
e) Signal transduction inhibitors (STI) that selectively inhibit one or more vital steps in signaling pathways in the normal function of cancer cells, thereby leading to apoptosis, especially (i) bcr/abl kinase inhibitors (such as STI 571 (GLEEVEC®), Dasatinib, Ponatinib, Imatinib, Nilotinib, Radotinib); (ii) inhibitors of Akt family kinases or the Akt pathway (such as Sirolimus, Everolimus, Temsirolimus); (iii) cell cycle kinase inhibitors (such as Palbociclib, Ribociclib, Abemaciclib); (iv) phosphatidyl inositol kinase inhibitors (such as Copanlisib, Duvelisib, Idelalisib, Leniolisib, Alpelisib, Umbralisib); (v) ALK inhibitors (such as Crizotinib, Ceritinib, Alectinib, Lorlatinib); (vi) BTK inhibitors (such as Ibrutinib, Acalabrutinib, Zanubrutinib); (vii) IDH inhibitors (such as Enasidenib, Ivosidenib); (viii) RET inhibitors (such as Selpercatinib); (ix) BCL-2 inhibitors (such as Venetoclax); or (x) c-MET inhibitors (such as Capmatinib, Tepotinib);
f) Angiogenesis inhibitors, especially VEGF signalling inhibitors (such as Bevacuzimab (Avastin), Ramucirumab, Sorafenib, Axitinib);
g) Poly-ADP-Ribose-Polymerase (PARP) inhibitors (such as Olaparib, Niraparib, Rucaparib, Talazoparib);
h) Proteasome inhibitors (such as Bortezomib, Carfilzomib, Ixazomib);
i) HDAC inhibitors (such as Vorinostat, Romidepsin, Belinostat, Panobinostat, Tucidinostat);
j) EZH2 inhibitors (such as Tazemetostat);
k) Immune Checkpoint inhibitors (for example: anti-PD1 antibodies such as Pembrolizumab (Lambrolizumab, MK-3475), Nivolumab, Pidilizumab (CT-011), AMP-514/MED10680, PDR001, SHR-1210, REGN2810, BGBA317, PF-06801591, MGA-012, TSR042, JS-001, BCD100, IBI-308, BI-754091, Toripalimab; fusion proteins targeting PD-1 such as AMP-224; small molecule anti-PD1 agents such as compounds disclosed in WO2015/033299, WO2015/044900 and WO2015/034820; anti-PD1L antibodies, such as BMS-936559, Atezolizumab (MPDL3280A, RG7446), Avelumab (MSB0010718C), Durvalumab (MEDI4736); anti-PDL2 antibodies, such as AMP224; anti-CTLA-4 antibodies, such as Ipilimumab, Tremelmumab; anti-Lymphocyte-activation gene 3 (LAG-3) antibodies such as Relatlimab (BMS-986016), IMP701, IMP731, MK-4280, ImmuFact IMP321; anti-T cell immunoglobulin mucin-3 (TIM-3) antibodies, such as MBG453, TSR-022; anti-T cell immunoreceptor with Ig and ITIM domains (TIGIT) antibodies, such as RG6058 (anti-TIGIT, MTIG7192A); anti-Killer-cell immunoglobulin-like receptors (KIR) antibodies such as Lirilumab (IPH2102/BMS-986015); antagonists of Galectins (such as of Galectin-1, Galectin-9); anti-B and T lymphocyte attenuator (BTLA) antibodies such as JS004;

l) Vaccination approaches (such as dendritic cell vaccination, DNA, RNA, peptide or protein vaccination (for example with gp 100 peptide or MAGE-A3 peptide));
m) Re-introduction of patient derived or allogenic (non-self) cancer cells genetically modified to secrete immunomodulatory factors such as granulocyte monocyte colony stimulating factor (GMCSF), gene-transfected tumor cell vaccine (GVAX) or Fms-related tyrosine kinase 3 (Flt-3) ligand gene-transfected tumor cell vaccine (FVAX), or Toll like receptor enhanced GM-CSF tumor based vaccine (TEGVAX);
n) T-cell based adoptive immunotherapies, including chimeric antigen receptor (CAR) engineered T-cells (such as CTL019);
o) Cytokine or immunocytokine based therapy (such as Interferon alpha, interferon beta, interferon gamma, interleukin 2, interleukin 6, interleukin 10, interleukin 15, TGF-β);
p) Toll-like receptor (TLR) agonists (such as resiquimod, imiquimod, glucopyranosyl lipid A, CpG oligodesoxynucleotides);
q) Thalidomide analogues (such as Lenalidomide, Pomalidomide);
r) Indoleamin-2,3-Dioxgenase (IDO) and/or Tryptophane-2,3-Dioxygenase (TDO) inhibitors (such as RG6078/NLG919/GDC-0919; Indoximod/1MT (1-methyltryptophan), INCB024360/Epacadostat, PF-06840003 (EOS200271), F001287);
s) Activators of T-cell co-stimulatory receptors (for example anti-CD137/4-1BB antibodies, such as BMS-663513/Urelumab, Utomilumab (PF-05082566), and second generation 4-1BB agonistic drugs (Claus et al., MABS 2023, VOL. 15 (1), 2167189)); anti-OX40/CD134 (Tumor necrosis factor receptor superfamily, member 4) antibodies (including agonist antibodies) and fusion proteins, such as RG7888 (MOXR0916), 9B12, MEDI6469, GSK3174998, MEDI6383, MEDI0562), anti-OX40-Ligand/CD252; anti-glucocorticoid-induced TNFR family related gene (GITR) antibodies and fusion proteins (such as TRX518, MEDI1873, MK-4166, BMS-986156, BMS-986153); anti-CD40 (TNF receptor superfamily member 5) antibodies (such as Dacetuzumab (SGN-40), HCD122, CP-870,893, RG7876, ADC-1013, APX005M, SEA-CD40); anti-CD40-Ligand antibodies (such as BG9588); anti-CD27 antibodies (such as Varlilumab); anti-CD28 antibodies; anti-ICOS antibodies;
t) Molecules binding a tumor specific antigen as well as a T-cell surface marker such as bispecific antibodies (for example RG7802 targeting CEA and CD3) or antibody fragments; antibody mimetic proteins such as designed ankyrin repeat proteins (DARPINS), bispecific T-cell engager (BITE, for example Blinatumomab, Amivantanab, Tebentafusp, and more in clinical development (Wei et al., Frontiers in Immunology, 2022, 13:1035276);
u) Antibodies or small molecular weight inhibitors targeting colony-stimulating factor-1 receptor (CSF-1R) (for example Emactuzumab (RG7155), Cabiralizumab (FPA-008), PLX3397);
v) Agents targeting immune cell check points on natural killer cells such as antibodies against Killer-cell immunoglobulin-like receptors (KIR) for example Lirilumab (IPH2102/BMS-986015);
w) Agents targeting the Adenosine receptors or the ectonucleases CD39 and CD73 that convert ATP to Adenosine, such as MEDI9447 (anti-CD73 antibody), PBF-509, CPI-444 (Adenosine A2a receptor antagonist);
x) Antagonists of chemokine receptors including CCR2 or CCR4;
y) Agents that deplete or inhibit T regulatory cells (for example anti-CD25 monoclonal antibodies (such as Daclizumab) or by ex vivo anti-CD25 bead depletion) or that reverse/prevent T cell anergy or exhaustion
z) Antibody or biologic-based disease-modifying antirheumatic drugs (DMARDS) useful for the treatment of autoimmune diseases such as antibodies (including modified antibodies); biologics targeting CD20 (for example Rituximab), CD3 (for example Teplizumab), TNF alpha (for example Etanercept), IFN alpha (for example Anifrolumab), CD80 or CD86 (for example Abatacept), VLA-4 (for example Natalizumab), IL-1 (for example Anakinra), IL-6 (for example Sarilumab), IL-17 (for example Secukinumab), IL-23 (for example Guselkumab), or IL12/23 (for example Ustekinumab); small molecule DMARDS such as methotrexate, leflunomide, hydroxychloroquine, sulfasalazine, or Janus kinase (JAK) inhibitors;
bb) Glucocorticoids;
cc) Antibody-drug conjugates, such as Mirvetuximab soravtansine, Tisotumab vedotin-tftv, Loncastuximab tesirine-lpyl, Belantamab mafodotin-blmf, Sacituzumab govitecan, Trastuzumab deruxtecan, Enfortumab vedotin, Polatuzumab vedotin-piiq, Moxetumomab pasudotox, Inotuzumab ozogamicin, Trastuzumab emtansine, Brentuximab vedotin, or Gemtuzumab ozogamicin;
dd) Radioligand therapies such as 177Lu-Lutathera or 177Lu-PSMA-617.

The term “chemotherapy” refers to the treatment of cancer with one or more cytotoxic anti-neoplastic agents (“cytotoxic chemotherapy agents”). Chemotherapy is often used in conjunction with other cancer treatments, such as radiation therapy or surgery. The term especially refers to conventional chemotherapeutic agents which act by killing cells that divide rapidly, one of the main properties of most cancer cells. Chemotherapy may use one drug at a time (single-agent chemotherapy) or several drugs at once (combination chemotherapy or polychemotherapy). Chemotherapy using drugs that convert to cytotoxic activity only upon light exposure is called photochemotherapy or photodynamic therapy.

The term “cytotoxic chemotherapy agent” or “chemotherapy agent” as used herein refers to an active anti-neoplastic agent inducing apoptosis or necrotic cell death. When used in combination with the COMPOUND 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol, especially with the COMPOUND adipic acid salt in crystalline form A, the term especially refers to conventional cytotoxic chemotherapy agents such as:
a) alkylating agents (for example mechlorethamine, chlorambucil, cyclophosphamide, ifosfamide, streptozocin, carmustine, lomustine, melphalan, busulfan, dacarbazine, temozolomide, thiotepa or altretamine; in particular temozolomide);
b) platinum drugs (for example cisplatin, carboplatin or oxaliplatin);
c) antimetabolite drugs (for example 5-fluorouracil, capecitabine, 6-mercaptopurine, methotrexate, gemcitabine, cytarabine, fludarabine or pemetrexed);
d) anti-tumor antibiotics (for example daunorubicin, doxorubicin, epirubicin, idarubicin, actinomycin-D, bleomycin, mitomycin-C or mitoxantrone);
e) mitotic inhibitors (for example paclitaxel, docetaxel, ixabepilone, vinblastine, vincristine, vinorelbine, vindesine or estramustine); or
f) topoisomerase inhibitors (for example etoposide, teniposide, topotecan, irinotecan, diflomotecan or elomotecan).

When used in combination with the COMPOUND 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol, especially with the COMPOUND adipic acid salt in crystalline form A, preferred cytotoxic chemotherapy agents are the above-mentioned alkylating agents (notably mechlorethamine, chlorambucil, cyclophosphamide, ifosfamide, streptozocin, carmustine, lomustine, melphalan, busulfan, dacarbazine, 3-methyl-(triazen-1-yl) imidazole-4-carboxamide (MTIC) and prodrugs thereof such as especially temozolomide, thiotepa, altretamine; or pharmaceutically acceptable salts of these compounds; in particular temozolomide); and mitotic inhibitors (notably paclitaxel, docetaxel, ixabepilone, vinblastine, vincristine, vinorelbine, vindesine, estramustine; or pharmaceutically acceptable salts of these compounds; in particular paclitaxel). Most preferred cytotoxic chemotherapy agents to be used in combination with the COMPOUND 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol, especially with the COMPOUND adipic acid salt in crystalline form A are those routinely used in the treatment of glioblastoma multiforme, in particular temozolomide. Equally preferred is radiotherapy.

Chemotherapy may be given with a curative intent, or it may aim to prolong life or to palliate symptoms.
a) Combined modality chemotherapy is the use of drugs with other cancer treatments, such as radiation therapy or surgery.
b) Induction chemotherapy is the first line treatment of cancer with a chemotherapeutic drug. This type of chemotherapy is used for curative intent.
c) Consolidation chemotherapy is the given after remission in order to prolong the overall disease-free time and improve overall survival. The drug that is administered is the same as the drug that achieved remission.
d) Intensification chemotherapy is identical to consolidation chemotherapy but a different drug than the induction chemotherapy is used.
e) Combination chemotherapy involves treating a patient with a number of different drugs simultaneously. The drugs differ in their mechanism and side effects. The biggest advantage is minimizing the chances of resistance developing to any one agent. Also, the drugs can often be used at lower doses, reducing toxicity.
f) Neoadjuvant chemotherapy is given prior to a local treatment such as surgery and is designed to shrink the primary tumor. It is also given to cancers with a high risk of micro-metastatic disease.
g) Adjuvant chemotherapy is given after a local treatment (radiotherapy or surgery). It can be used when there is little evidence of cancer present, but there is risk of recurrence. It is also useful in killing any cancerous cells that have spread to other parts of the body. These micro-metastases can be treated with adjuvant chemotherapy and can reduce relapse rates caused by these disseminated cells.
h) Maintenance chemotherapy is a repeated low-dose treatment to prolong remission.
i) Salvage chemotherapy or palliative chemotherapy is given without curative intent, but simply to decrease tumor load and increase life expectancy. For these regimens, a better toxicity profile is generally expected.

When used in combination with the COMPOUND 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol, especially with the COMPOUND adipic acid salt in crystalline form A, preventive or curative forms of chemotherapy (or mutatis mutandis: radiotherapy) such as those listed under a), b) c), d), e), and especially g) and/or h) above are preferred.

When used in combination with the COMPOUND 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol, especially with the COMPOUND adipic acid salt in crystalline form A, chemotherapy, radiotherapy, EGFR inhibitors, aromatase inhibitors, and immunotherapy are preferred. Immunotherapy such as especially those targeting the programmed cell death receptor 1 (PD-1 receptor) or its ligand PD-L1 i.e. PD1 and/or PDL1 blockade; and/or CTLA4 blockade; and/or TIGIT blockade and/or LAG3 blockade; or other targeted therapies are especially preferred. Most preferred immunotherapy used in combination with the COMPOUND 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol, especially with the COMPOUND adipic acid salt in crystalline form A, is PD1 and/or PDL1 blockade.

In some embodiments, the crystalline form of the present invention is administered in an amount of between about 1 mg and about 1000 mg per day, for example, between about 5 mg and about 500 mg per day, about 25 mg and about 400 mg per day, or about 50 mg and about 200 mg per day.

The term “prevention” as used herein may refer to prophylaxis.

The present application provides deuterated forms of COMPOUND, pharmaceutically acceptable salts thereof, and their use in the treatment or prevention of various diseases or disorders ameliorated by modulating chemokine receptor 6 (CCR6).
14) Another aspect of the present invention relates to a deuterated form of COMPOUND, which is also a compound of Formula (II):

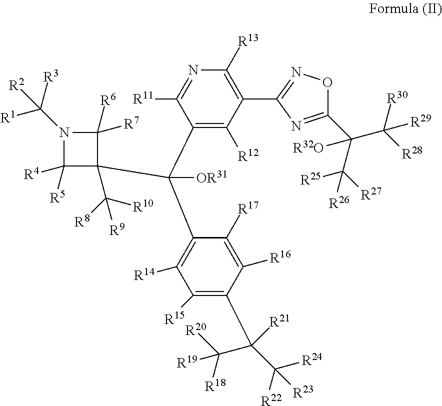

or a pharmaceutically acceptable salt thereof,
wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, and R³², are each independently selected from hydrogen and deuterium; and
wherein at least one R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, and R³², represents deuterium.

With regard to the compounds of Formula (II), when a particular atomic position is designated as representing deuterium or “D” or “d”, it is understood that the abundance of deuterium at that position is substantially greater than the natural abundance of deuterium, which is about 0.015%. A position designated as having deuterium typically has a minimum isotopic enrichment factor of, in certain embodiments, at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium position.

The term “pharmaceutically acceptable salts” refers to salts that retain the desired biological activity of the subject compound and exhibit minimal undesired toxicological effects. Such salts include inorganic or organic acid and/or base addition salts depending on the presence of basic and/or acidic groups in the subject compound. For reference see for example “Handbook of Pharmaceutical Salts. Properties, Selection and Use.”, P. Heinrich Stahl, Camille G. Wermuth (Eds.), Wiley-VCH, 2008; and “Pharmaceutical Salts and Co-crystals”, Johan Wouters and Luc Quéré(Eds.), RSC Publishing, 2012.

The metabolism of COMPOUND was investigated in liver microsomes and hepatocytes of man and a set of animal species (mouse, rat, dog, rabbit, and cynomolgus monkey) by using the 14C-labeled analog of COMPOUND: (R)-2-(3-(5-((1,3-dimethylazetidin-3-yl)(hydroxy)(4-isopropylphenyl)methyl)pyridin-3-yl)-1,2,4-oxadiazol-5-yl-3-¹⁴C) propan-2-ol. As a result, several metabolites of COMPOUND could be observed and proposals for their chemical structures were made. Certain positions in the molecule of COMPOUND were found to be metabolically relevant. In particular, such positions are the isopropyl substituent of the phenyl ring, and especially the two methyl substituents of the azetidinyl ring and the 2-hydroxy-propan-2-yl substituent of the oxadiazolyl ring. The substitution at one or more metabolically relevant position of COMPOUND with one or more deuterium atoms may therefore afford at least one therapeutic advantage resulting from greater metabolic stability, for example, increased in vivo half-life or reduced dosage requirements, or may lead to an improved safety profile. Thus, the deuterated analogs of COMPOUND as recited in the embodiments 15) to 19) are especially preferred.
15) One embodiment relates to a compound according to embodiment 14), wherein at least one, especially all, of R¹, R², and R³ represent deuterium; and wherein R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, and R³² represent hydrogen;
16) Another embodiment relates to a compound according to embodiment 14), wherein at least one, especially all, of R⁸, R⁹, and R¹⁰ represent deuterium; and wherein R¹, R², R³, R⁴, R⁵, R⁶, R⁷, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, and R³² represent hydrogen;
17) Another embodiment relates to a compound according to embodiment 14), wherein at least one, especially all, of R²⁵, R²⁶, R²⁷, R²⁸, R²⁹ and R³⁰ represents deuterium; and wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R³¹, and R³², represents hydrogen.
18) Another embodiment relates to a compound according to embodiment 14), wherein at least one, especially all, of R¹⁸, R¹⁹, R²⁰, R²¹, R²², R²³, and R²⁴ represents deuterium; and wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R²⁵, R²⁶, R²⁷, R²⁸, R²⁹, R³⁰, R³¹ and R³², represents hydrogen.
19) Another embodiment relates to a compound according to embodiment 14) which is
(R)-2-(3-(5-(hydroxy (4-isopropylphenyl) (3-methyl-1-(methyl-d)azetidin-3-yl)methyl)pyridin-3-yl)-1,2,4-oxadiazol-5-yl)propan-2-ol;
(R)-2-(3-(5-(hydroxy (4-isopropylphenyl) (3-methyl-1-(methyl-d2)azetidin-3-yl)methyl)pyridin-3-yl)-1,2,4-oxadiazol-5-yl)propan-2-ol;
(R)-2-(3-(5-(hydroxy (4-isopropylphenyl) (3-methyl-1-(methyl-d3)azetidin-3-yl)methyl)pyridin-3-yl)-1,2,4-oxadiazol-5-yl)propan-2-ol;
(R)-2-(3-(5-(hydroxy (4-isopropylphenyl) (1-methyl-3-(methyl-d)azetidin-3-yl)methyl)pyridin-3-yl)-1,2,4-oxadiazol-5-yl)propan-2-ol;
(R)-2-(3-(5-(hydroxy (4-isopropylphenyl) (1-methyl-3-(methyl-d2)azetidin-3-yl)methyl)pyridin-3-yl)-1,2,4-oxadiazol-5-yl)propan-2-ol;
(R)-2-(3-(5-(hydroxy (4-isopropylphenyl) (1-methyl-3-(methyl-d3)azetidin-3-yl)methyl)pyridin-3-yl)-1,2,4-oxadiazol-5-yl)propan-2-ol;
2-(3-(5-((R)-(1,3-dimethylazetidin-3-yl)(hydroxy)(4-isopropylphenyl)methyl)pyridin-3-yl)-1,2,4-oxadiazol-5-yl)propan-1-d-2-ol;
2-(3-(5-((R)-(1,3-dimethylazetidin-3-yl)(hydroxy)(4-isopropylphenyl)methyl)pyridin-3-yl)-1,2,4-oxadiazol-5-yl)propan-1,1-d2-2-ol;
2-(3-(5-((R)-(1,3-dimethylazetidin-3-yl)(hydroxy)(4-isopropylphenyl)methyl)pyridin-3-yl)-1,2,4-oxadiazol-5-yl)propan-1,1,1-d3-2-ol;
2-(3-(5-((R)-(1,3-dimethylazetidin-3-yl)(hydroxy)(4-isopropylphenyl)methyl)pyridin-3-yl)-1,2,4-oxadiazol-5-yl)propan-1,1,1,3-d4-2-ol;
2-(3-(5-((R)-(1,3-dimethylazetidin-3-yl)(hydroxy)(4-isopropylphenyl)methyl)pyridin-3-yl)-1,2,4-oxadiazol-5-yl)propan-1,1,1,3,3-d5-2-ol;
(R)-2-(3-(5-((1,3-dimethylazetidin-3-yl)(hydroxy)(4-isopropylphenyl)methyl)pyridin-3-yl)-1,2,4-oxadiazol-5-yl)propan-1,1,1,3,3,3-d6-2-ol;
2-(3-(5-((1R)-(1,3-dimethylazetidin-3-yl)(hydroxy)(4-(propan-2-yl-1-d)phenyl)methyl)pyridin-3-yl)-1,2,4-oxadiazol-5-yl)propan-2-ol;
2-(3-(5-((1R)-(1,3-dimethylazetidin-3-yl)(hydroxy)(4-(propan-2-yl-1,1-d2)phenyl)methyl)pyridin-3-yl)-1,2,4-oxadiazol-5-yl)propan-2-ol;
2-(3-(5-((1R)-(1,3-dimethylazetidin-3-yl)(hydroxy)(4-(propan-2-yl-1,1,1-d3)phenyl)methyl)pyridin-3-yl)-1,2,4-oxadiazol-5-yl)propan-2-ol;
2-(3-(5-((1R)-(1,3-dimethylazetidin-3-yl)(hydroxy)(4-(propan-2-yl-1,1,1,3-d4)phenyl)methyl)pyridin-3-yl)-1,2,4-oxadiazol-5-yl)propan-2-ol;
2-(3-(5-((1R)-(1,3-dimethylazetidin-3-yl)(hydroxy)(4-(propan-2-yl-1,1,1,3,3-d5)phenyl)methyl)pyridin-3-yl)-1,2,4-oxadiazol-5-yl)propan-2-ol;
2-(3-(5-((1R)-(1,3-dimethylazetidin-3-yl)(hydroxy)(4-(propan-2-yl-1,1,1,3,3,3-d6)phenyl)methyl)pyridin-3-yl)-1,2,4-oxadiazol-5-yl)propan-2-ol; or
2-(3-(5-((1R)-(1,3-dimethylazetidin-3-yl)(hydroxy)(4-(propan-2-yl-d7)phenyl)methyl)pyridin-3-yl)-1,2,4-oxadiazol-5-yl)propan-2-ol;
   or a pharmaceutically acceptable salt thereof.

The present disclosure provides synthetic methods for incorporating deuterium into COMPOUND. Synthetic methods for incorporating radioisotopes into organic compounds are well known in the art (Deuterium Labeling in Organic Chemistry by Alan F. Thomas et al. 1971; The Renaissance of H/D Exchange by Jens Atzrodt et al., Angew. Chem. Int. Ed. 2007, 7744-7765; The Organic Chemistry of Isotopic Labelling by James R. et al., 2011), and the skilled person in the art will readily recognize the methods applicable for COMPOUND. Notably, deuterated compounds of Formula (II) may be prepared in analogy to the methods described in WO2021219849 but using the appropriate isotopic variation of suitable reagents or starting materials. More particularly, compounds of Formula (II) may be prepared according to the Scheme 1:

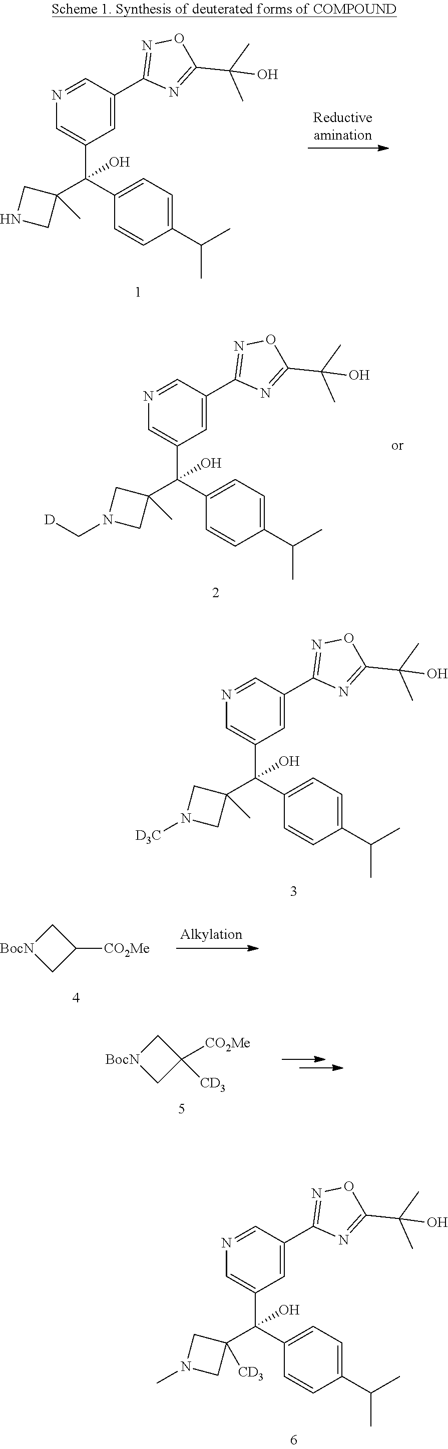 [SMILES: CC(C)c1ccc([C@](O)(c2cncc(-c3noc(C(C)(C)O)n3)c2)C2(C)CNC2)cc1 ; [2H]CN1CC(C)([C@](O)(c2ccc(C(C)C)cc2)c2cncc(-c3noc(C(C)(C)O)n3)c2)C1 ; [2H]C([2H])([2H])N1CC(C)([C@](O)(c2ccc(C(C)C)cc2)c2cncc(-c3noc(C(C)(C)O)n3)c2)C1 ; COC(=O)C1CN(C(=O)OC(C)(C)C)C1 ; [2H]C([2H])([2H])C1(C(=O)OC)CN(C(=O)OC(C)(C)C)C1 ; [2H]C([2H])([2H])C1([C@](O)(c2ccc(C(C)C)cc2)c2cncc(-c3noc(C(C)(C)O)n3)c2)CN(C)C1]

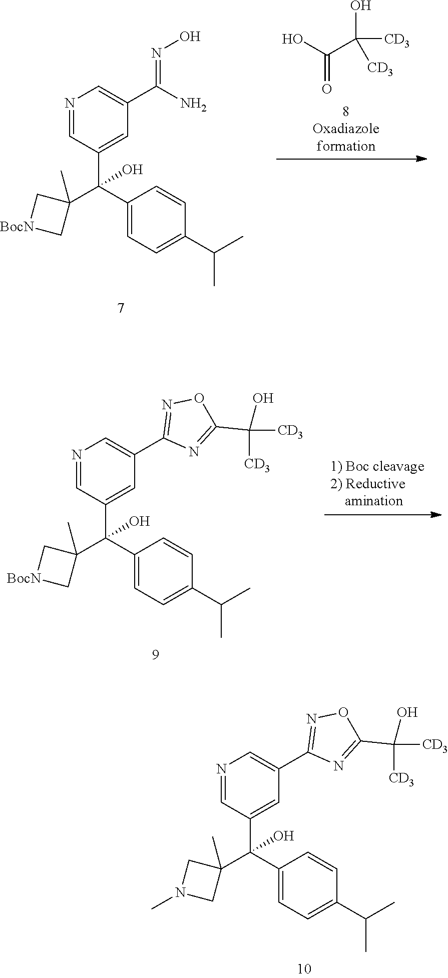 [SMILES: CC(C)c1ccc([C@](O)(c2cncc(/C(N)=N/O)c2)C2(C)CN(C(=O)OC(C)(C)C)C2)cc1 ; [2H]C([2H])([2H])C(O)(C(=O)O)C([2H])([2H])[2H] ; [2H]C([2H])([2H])C(O)(c1nc(-c2cncc([C@@](O)(c3ccc(C(C)C)cc3)C3(C)CN(C(=O)OC(C)(C)C)C3)c2)no1)C([2H])([2H])[2H] ; [2H]C([2H])([2H])C(O)(c1nc(-c2cncc([C@@](O)(c3ccc(C(C)C)cc3)C3(C)CN(C)C3)c2)no1)C([2H])([2H])[2H]]

In the scheme above, substitution of the azetidine nitrogen of intermediate 1 (described in WO2021219849) can give rise to two possible deuterated forms (2 or 3): hence, a CD3 group can be introduced via reductive amination using paraformaldehyde-d2 in presence of NaBD₄ in 2,2,2-trifluoroethanol-d3 and following conditions described in [M. Tajbakhsh et al. Synthesis, 2011, 3, 490]. Alternatively, a CDH₂ group can be introduced similarly via reductive amination of 1 using a non-deuterated formaldehyde solution and NaBHD₄ as described in the Examples of the present application.

Alkylation of methyl 1-Boc-azetidine-3-carboxylate (4) with CD₃I as previously reported with CH₃I in the patent literature (WO2006073361 or WO2021102314) can provide intermediate 5 which can be converted to the final compound 6 using procedures described in WO2021219849. PyBOP-mediated oxadiazole formation of intermediate 7 (described in WO2021219849) using 2-hydroxy-2-methyl-d3-propanoic-3,3,3-d3 acid (8) provides intermediate 9 which is subjected to Boc cleavage and reductive amination to give the hexadeuterated compound 10. Acid 8 is obtained from acetone-d6 in two steps as described in published procedures [D. Passarella et al. J. Labelled Cpd. Radiopharm. 1999, 42, 275 and U.S. Pat. No. 4,286,105].

### Preparation of Deuterated Analogs of COMPOUND:

### (R)-2-(3-(5-(hydroxy(4-isopropylphenyl)(3-methyl-1-(methyl-d)azetidin-3-yl)methyl)pyridin-3-yl)-1,2,4-oxadiazol-5-yl)propan-2-ol

To a solution of the secondary amine 1 (Scheme 1 hereinabove) as HCl salt described in WO2021219849 (20 mg, 0.04 mmol, 1 eq) in trifluoroethanol (200 UL, 0.2 M) was sequentially added at RT, Et₃N (40 μL, 0.285 mmol, 7.1 eq) and a 37% solution of formaldehyde in H₂O (12 μL, 0.161 mmol, 4 eq). The mixture was stirred for 2 min at RT and NaBD₄ (4 mg, 0.086 mmol, 2.1 eq) was added at RT. The reaction mixture was stirred for 2 h at RT to reach completion, diluted with CH₂Cl₂ and quenched with a sat. solution of NaHCO₃. The aq. phase was extracted twice with CH₂Cl₂, and the combined organic phases were dried over MgSO₄ and concentrated in vacuo. The crude was purified by Prep LC-MS (basic conditions) to provide (R)-2-(3-(5-(hydroxy (4-isopropylphenyl) (3-methyl-1-(methyl-d)azetidin-3-yl)methyl)pyridin-3-yl)-1,2,4-oxadiazol-5-yl)propan-2-ol as white foam (12 mg, 69% yield). LC-MS (I): t_{R}=0.70 min; [M+H⁺]: 438.16. ¹H NMR (400 MHz, d6-DMSO) δ: 9.05 (s, 1H), 8.50 (s, 1H), 8.24 (s, 1H), 7.23 (d, 2H), 7.11 (d, J=7.9 Hz, 2H), 6.13 (s, 2H), 3.41-3.49 (m, 2H), 3.02 (dd, J₁=72.5 Hz, J₂=6.4 Hz, 2H), 2.85-2.90 (m, 1H), 2.10 (s, 2H), 1.61 (s, 6H), 1.38 (s, 3H), 1.20 (d, J=6.9 Hz, 6H).

### (R)-2-(3-(5-((1,3-dimethylazetidin-3-yl)(hydroxy)(4-isopropylphenyl)methyl)pyridin-3-yl)-1,2,4-oxadiazol-5-yl)propan-1,1,1,3,3,3-d6-2-ol

Tert-butyl (R)-3-(hydroxy (5-(5-(2-hydroxypropan-2-yl-1,1,1,3,3,3-d6)-1,2,4-oxadiazol-3-yl)pyridin-3-yl) (4-isopropylphenyl)methyl)-3-methylazetidine-1-carboxylate (Intermediate 9) was prepared as follows: To a suspension of intermediate 7 described in WO2021219849 (1267 mg, 2.79 mmol, 1 eq), 2-hydroxy-2-methyl-d3-propanoic-3,3,3-d3 acid 8 (614 mg, 5.57 mmol, 2 eq), DIPEA (1.43 mL, 8.36 mmol, 3 eq) and K₃PO₄ (2415 mg, 11.1 mmol, 4 eq) in DMF (10 mL) was added PyBOP (3191 mg, 6.13 mmol, 2.2 eq) and the mixture was stirred at 90° C. for 3 h. More 2-hydroxy-2-methyl-d3-propanoic-3,3,3-d3 acid 8 (130 mg, 1.11 mmol, 0.4 eq) and PyBOP (638.2 mg, 1.23 mmol, 0.44 eq) were added, and stirring was continued at 90° C. for another 3 h. The reaction mixture was filtered to remove solid particles and the filtrate directly injected into Prep LC-MS (acidic) to give 725 mg of 9 as white solid. LC-MS (I): t_{R}=1.04 min, [M+H⁺]: 529.21.

To a solution of intermediate 9 (725 mg, 1.37 mmol, 1 eq) in dioxane (5 mL) was added dropwise HCl 4M in dioxane (3.5 mL, 14 mmol, 10.2 eq) and the reaction mixture was stirred at RT of 6 h. The solvent was evaporated under reduced pressure and the crude dried under HV overnight (685 mg, white solid). LC-MS analysis (I): t_{R}=0.68 min, [M+H⁺] 429.29.

To a suspension of the crude obtained in the preceding step (637 mg, 1.37 mmol, 1 eq) in THF (5 mL) was added DIPEA (0.704 mL, 4.11 mmol, 3 eq) dropwise, followed by formaldehyde 37 wt % in H₂O (0.204 mL, 2.74 mmol, 2 eq) and sodium triacetoxyborohydride (581 mg, 2.6 mmol, 1.9 eq). The reaction mixture was stirred at RT for 15 min. The solid particles were filtered off and THF was evaporated under reduced pressure. Purification by Prep LC-MS (acidic conditions followed by basic conditions) provided (R)-2-(3-(5-((1,3-dimethylazetidin-3-yl)(hydroxy)(4-isopropylphenyl)methyl)pyridin-3-yl)-1,2,4-oxadiazol-5-yl)propan-1,1,1,3,3,3-d6-2-ol (10) as white solid (494 mg). LC-MS (I): t_{R}=0.70 min, [M+H⁺]: 443.24.
20) Another embodiment relates to a pharmaceutical composition comprising a compound according to any one of embodiments 14) to 19), and at least one pharmaceutically acceptable carrier material; especially such as especially a carrier material selected from embodiment 9).

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of Formula (II), or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

The compounds of Formula (II) are useful as medicaments; in particular in the treatment of cancer; and inflammatory/autoimmune diseases or disorders such as those disclosed for COMPOUND and/or the crystalline form of COMPOUND adipic acid salt of the current application.

For avoidance of any doubt, if a compound of Formula (II) or a pharmaceutically acceptable salt thereof, is described as useful for the prevention or treatment of certain diseases or disorders, such a compound of Formula (II) or a pharmaceutically acceptable salt thereof is likewise suitable for use in the preparation of a medicament for the prevention or treatment of said diseases.

For avoidance of any doubt, if a compound of Formula (II) or a pharmaceutically acceptable salt thereof, is described as useful for the prevention or treatment of certain diseases, such a compound of Formula (II) or a pharmaceutically acceptable salt thereof is likewise suitable for use in a method for the prevention or treatment of a disease or disorder, said method comprising administering to a subject in need of such prevention or treatment an effective amount of said deuterated forms of COMPOUND or pharmaceutically acceptable salts thereof.

In some embodiments, a compound of Formula (II) or a pharmaceutically acceptable salt thereof is administered in an amount of between about 1 mg and about 1000 mg per day, for example, between about 5 mg and about 500 mg per day, about 25 mg and about 400 mg per day, or about 50 mg and about 200 mg per day.

### EXPERIMENTAL PART

All temperatures are stated in ° C.

ABBREVIATIONS (as used hereinbefore or hereinafter):

| | |
|---|---|
| Ac | Acetyl |
| aq. | Aqueous |
| Boc | tert.-butyloxycarbonyl |
| DIPEA | N-ethyldiisopropylamine |
| DMF | Dimethylformamide |
| DMSO | Dimethyl sulfoxide |
| eq. | Equivalent(s) |
| g | Gram(s) |
| h | Hour(s) |
| HV | High vacuo |
| HPLC | High performance liquid chromatography |
| ¹H-NMR | Proton nuclear magnetic resonance |
| LC | Liquid chromatography |
| K | Kelvin |
| kV | Kilovolt(s) |
| M | Molarity [mol/L] |
| mA | Milliampere(s) |
| mbar | Millibar(s) |
| Me | methyl |
| mg | Milligram(s) |
| min | Minute(s) |
| mL | Milliliter(s) |
| mm | Millimeter(s) |
| mmol | Millimole(s) |
| NMR | Nuclear magnetic resonance spectroscopy |
| MS | Mass spectrometry |
| NEt₃ | Triethylamine |
| org. | Organic |
| Prep | Preparative |
| PyBOP | (benzotriazol-1-yloxy)tripyrrolidinophosphonium |
| | hexafluorophosphate |
| RH | Relative humidity |
| r.t. or RT | Room temperature |
| rpm | Revolutions per minute |
| s | Second(s) |
| sat. | Saturated |
| tBME (or TBME) | Tertiary-butyl methyl ether |
| THF | Tetrahydrofuran |
| t_{R} | retention time |
| w/w | Weight per weight |

### Purification and Characterization Methods

### Liquid Chromatography-Mass Spectrometry (LC-MS)

The LC-MS retention times have been obtained using the following elution conditions: LC-MS (I): Zorbax RRHD SB-Aq, 1.8 μm, 2.1×50 mm column thermostated at 40° C. The two elution solvents were as follows: solvent A=water+0.04% TFA; solvent B=MeCN. The eluent flow rate was 0.8 mL/min and the characteristics of the eluting mixture proportion in function of the time t (min) from start of the elution are summarized below (a linear gradient being used between two consecutive time points):

| t (min) | 0 | 1.2 | 1.9 | 2.1 |
|---|---|---|---|---|
| Solvent A (%) | 95 | 5 | 5 | 95 |
| Solvent B (%) | 5 | 95 | 95 | 5 |

### Preparative Liquid Chromatography-Mass Spectrometry (Prep LC-MS)

The purifications by preparative LC-MS have been performed using the following conditions: A X-Bridge column (Waters C18, 30×75 mm or 50×150 mm, 10 μm) or an Agilent Zorbax (SB-Aq 30×75 mm 5 μm) column was used with a linear gradient of water/formic acid 0.5% (A) and MeCN (B) (acidic conditions) or water/0.5% ammonium hydroxide solution (25%) (A) and MeCN (B) (basic conditions).

### X-Ray Powder Diffraction (XRPD)

Bruker D8 Advance diffractometer with flipstick stage, Cu Ka radiation (40 kV, 40 mA), and 1D-linear LynxEye detector (Bruker AXS GmbH) was used. The sample was prepared on a silicon single crystal sample holder with a cavity of 25 mm diameter and 0.5 mm depth. It was spread with a microscope slide to obtain a flat surface. Diffractograms were collected in the reflection mode with coupled 2θ/θ angles in the range from 3-50° 20, an increment of 0.02° and an accumulation time of 0.4 s per step. The divergence slit was set to variable slit size and the antiscatter slit to 0.3°. The samples were continuously rotated with 30 rpm during the measurement.

The measuring software used was DIFFRAC Measurement Center V7.5.0 (release 2017, Bruker AXS GmbH) and the analyzing software—DIFFRAC.Suite Eva V4.2.2.3 (release 2016, Bruker AXS GmbH).

### Thermogravimetric Analyses (TGA)

Thermogravimetric analysis was performed with Mettler Toledo TGA/DSC 3+ STARe system Samples were weighed into aluminum pans with automatically perforated lid and heated from 30° C. to 350° C. with a scan rate of 10° C./min in a constant flow of nitrogen. The measurement and the data evaluation were performed with the software Mettler Toledo STARe version 16.00b.

### Gravimetric Vapor Sorption (GVS) Analysis

Gravimetric vapor sorption analysis was performed with SPS 100n (ProUmid GmbH & Co. KG). An appropriate amount of the sample was placed into a tared aluminum pan without any pre-treatment. The analysis was run at a temperature of 25° C. with the following humidity program: 40%-0%-95%-40% in steps of 5% with maximum equilibration time of 24 h at each step. Analyzing software was ProUmid Excel template #SPS_4_3_23_E2010_drycorr.xltm.

### Differential Scanning Calorimetry (DSC)

DSC data are collected on a Mettler Toledo DSC 3+ STARe system. Samples were prepared under ambient conditions. Aluminum sample pans from Mettler Toledo, part no. ME-51119870 and ME-51119873, automatically pierced by the instrument were used. A scan from −20° C. to 250° C. at 10° C./min was performed. The measurement and the data evaluation were performed with the software Mettler Toledo STARe version 16.00b.

### EXAMPLES

Amorphous material of COMPOUND may be prepared by the methods disclosed in WO2021219849.

### Example 1a

A suspension of 20.67 mg amorphous COMPOUND in 200 UL of a saturated solution of adipic acid in isopropanol is stirred at room temperature. The solid is filtered off after three weeks yielding COMPOUND adipic acid salt in crystalline form A as confirmed by XRPD. Proton nuclear magnetic resonance spectroscopy revealed that the solid contains the COMPOUND and adipic acid in equimolar amounts.

### Example 1b

102 mg of amorphous COMPOUND and 35 mg (1 eq.) adipic acid are suspended in 1 mL tBME. The mixture is stirred for three weeks at room temperature. The solid is filtered off and dried under vacuum, yielding 95 mg of white material of COMPOUND adipic acid salt in crystalline form A.

### Example 1c

500 mg amorphous COMPOUND and 172 mg (1 eq.) adipic acid are suspended in 5 mL acetone. The suspension is heated up. After cooling back to r.t., additional acetone is added to obtain again a stirrable suspension. The solid is filtered off after five days, providing 569 mg (85%) of COMPOUND adipic acid salt in crystalline form A.

### Example 1d

A 30 L double-jacketed glass-lined steel reactor was charged with adipic acid (277 g, 1.90 mol, 1.00 eq.) and acetone (6.7 L, 7 vol.). The reactor content was warmed to 45-50° C. and acetone solution containing COMPOUND (1.00 eq. COMPOUND, 7 vol. acetone) was added over 75-90 min at 45-50° C. to the adipic acid solution. After complete addition, the resulting suspension was cooled to 5-10° C. over 1 h and finally aged at this temperature for at least 1 h. The product was filtered off over a 30 L Buchi Nutsche with N₂-overpressure, washed three times with cold acetone (3×1.5 L), and finally dried on a rotary evaporator (900-10 mbar, 65° C.) to provide COMPOUND adipic acid salt in crystalline form A (856 g, 1.47 mmol, >99% w/w).

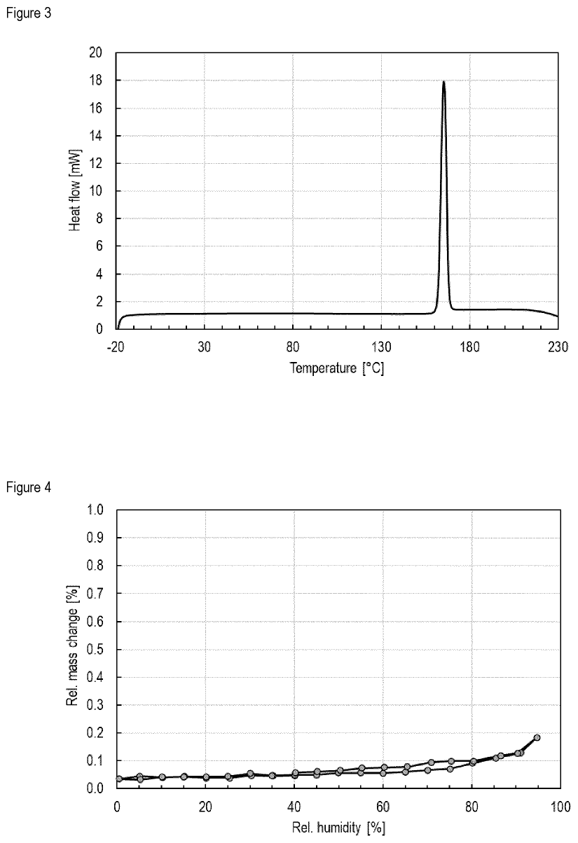

TABLE 1
Characterization data for COMPOUND adipic acid salt in crystalline form A

| Technique | Data Summary | Remarks |
|---|---|---|
| XRPD | Crystalline | FIG. 1 |
| DSC | Melting point 163.5 ± 2° C. | FIG. 3 |
| Hygroscopicity | Less than 0.1% water uptake between 40% and 80% RH. Sample | FIG. 4 |
| | is classified as non-hygroscopic acc. to Eur. Phar. | |
| Single Crystal | Single crystal X-ray diffraction at 160K elucidated a monoclinic | |
| | crystal system with the space group P 1 2₁ 1 and the following | |
| | parameters of the unit cell: a = 13.1342(6) Å, b = 9.1330(3) Å, c = | |
| | 14.2305(7) Å, α = 90°, β = 117.360(6), γ = 90°. Two molecules of | |
| | COMPOUND and two molecules of adipic acid are contained in | |
| | the unit cell. Stoichiometry 1:1 (i.e. mono adipate) is confirmed by | |
| | single crystal X-ray diffraction and ¹H-NMR. | |

### Example 2

Solid pharmaceutical compositions comprising COMPOUND adipic acid salt in crystalline form A, in dose strengths of 1 mg, 10 mg, and 50 mg API per unit of 250 mg, are shown in Table 2.

TABLE 2

| Pharmacopeial name | | | Quantity | Quantity | Quantity |
|---|---|---|---|---|---|
| (PhEur) | Material Name | Function | [w/w %] | [w/w %] | [w/w %] |
| Active pharmaceutical | COMPOUND adipic acid | Active | 0.53 | 5.34 | 26.70 |
| ingredient (API) | salt in crystalline form A | | | | |
| Colloidal Silicon | Aerosil 200 | Glidant | 0.50 | 0.50 | 0.50 |
| Dioxide | | | | | |
| Mannitol | Pearlitol 200 SD | Filler 1 | 69.47 | 64.66 | 43.30 |
| Microcrystalline | Avicel PH-200 | Filler 2 | 25.00 | 25.00 | 25.00 |
| Cellulose | | | | | |
| Croscarmellose | AcDiSol AD-711 | Disintegrant | 4.00 | 4.00 | 4.00 |
| Sodium | | | | | |
| Magnesium Stearate | Parteck LUB MST Emprove | Lubricant | 0.50 | 0.50 | 0.50 |
| Total | | | 100.00 | 100.00 | 100.00 |

The above-listed API, glidant, fillers, disintegrant, and lubricant are sieved and blended together (e.g. in a Turbula T10B™). The API is optionally blended with at least part of the glidant in a separate step preceding the addition of the rest of the excipients. The powder blend is then dry granulated (e.g. on a roller compactor Gerteis Mini Pactor®) and the resulting granulate is further blended before final encapsulation (e.g. BB6/S equipment) in hard gelatin capsules (e.g. ConiSnap® capsules, size 2) or in enteric coated capsules. The hard gelatin capsules may further be encapsulated in enteric coated capsules (so-called over-encapsulation) to potentially improve gastric tolerability.

### Reference Example 1

A solution of 51 mg of COMPOUND adipic acid salt in crystalline form A in 5 mL THF was evaporated at r.t. furnishing COMPOUND adipic acid salt in crystalline form B.

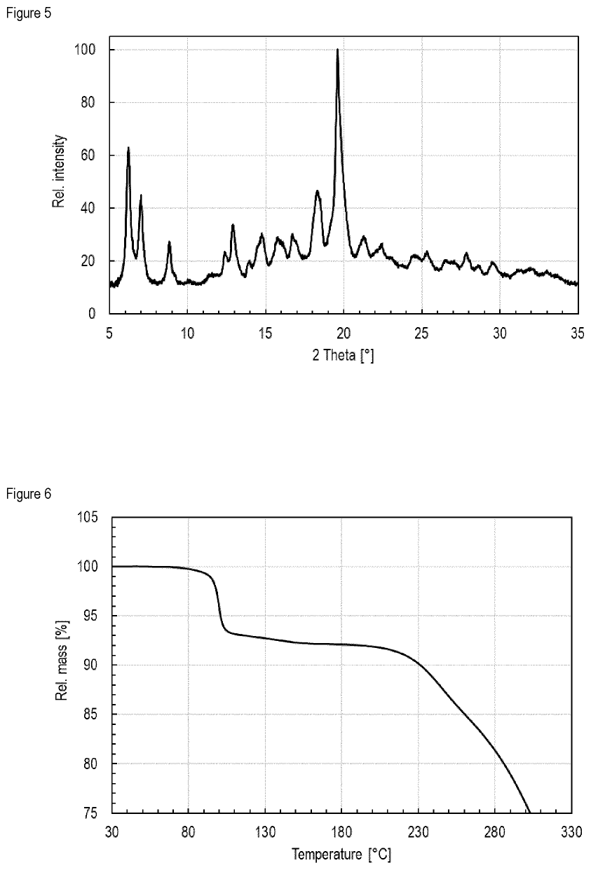

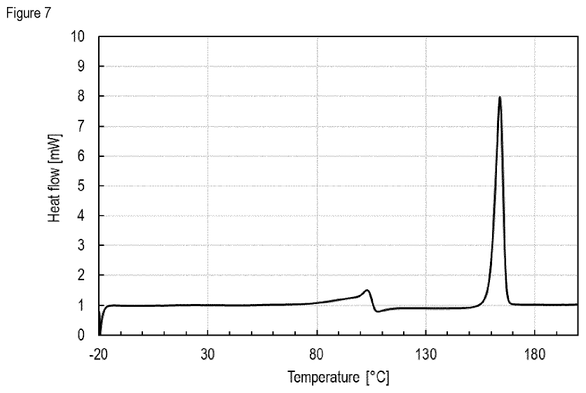

TABLE 3
Characterization data for COMPOUND adipic
acid salt in crystalline form B

| Technique | Data Summary | Remarks |
|---|---|---|
| XRPD | Crystalline | FIG. 5 |
| TGA | About 7% weight loss near 100° C. corresponding | FIG. 6 |
| | to the release of about 0.5 eq. of THF | |
| DSC | A broad endothermic peak near 100° C. | FIG. 7 |
| | attributable to the loss of solvent and | |
| | recrystallization, followed by melting of the | |
| | recrystallized form A | |

### Reference Example 2

Several crystallizations trials with different salt formers were performed, whereby COMPOUND was reacted with ascorbic acid, benzoic acid, butyric acid, citric acid, fumaric acid, glutamic acid, 1M HCl, DL-lactic acid, oxalic acid, 1M aq. H₃PO₄, succinic acid, 0.5M aq. H₂SO₄, or L-tartaric acid. Typically, mixtures of COMPOUND and the corresponding acid, optionally using isopropanol as a solvent, were stirred in closed vials at room temperature for about three months, and then analyzed. No formation of a solid salt of COMPOUND was observed with any of the above-mentioned salt formers.

1. A crystalline form of the compound 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol adipic acid salt, wherein the crystalline form is characterized by the presence of peaks in the X-ray powder diffractogram at the following angles of refraction 2θ: 6.9°, 17.8° and 19.2°.

2. A crystalline form of the compound 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol adipic acid salt according to claim 1, wherein the crystalline form is characterized by the presence of peaks in the X-ray powder diffractogram at the following angles of refraction 2θ: 6.9°, 12.2°, 16.8°, 17.8° and 19.2°.

3. A crystalline form of the compound 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol adipic acid salt according to claim 1, wherein the crystalline form is characterized by the presence of peaks in the X-ray powder diffractogram at the following angles of refraction 2θ: 6.9°, 12.2°, 15.6°, 16.4°, 16.8°, 17.8°, 19.2°, 20.3°, 21.5°, and 22.6°.

4. A crystalline form of the compound 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol adipic acid salt according to claim 1, wherein the crystalline form essentially shows the X-ray powder diffraction pattern as depicted in FIG. 1.

5. A crystalline form of the compound 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol adipic acid salt, wherein the crystalline form is characterized by the presence of an endothermic peak in the differential scanning calorimetry thermogram at 163.5±5° C.

6. A process for manufacturing of the crystalline form of the compound 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol adipic acid salt according to claim 1, said process comprising the following steps:
a) providing a first solution/suspension of about 1 eq. adipic acid in acetone;
b) providing a second solution comprising about 1 eq. of the compound 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol in acetone;
c) mixing of the first solution/suspension and the second solution, under heating;
d) cooling the resulting mixture;
e) optionally stirring the resulting mixture;
f) isolating the solid residue through a solid-liquid separation; and
g) drying the solid residue.

7. A process for manufacturing according to claim 6, wherein the mixing of the first solution/suspension and the second solution is performed by adding the second solution to the first solution/suspension, under heating.

8. A pharmaceutical composition comprising the crystalline form according to claim 1, and at least one pharmaceutically acceptable carrier material.

9. (canceled)

10. (canceled)

11. A method for the prevention or treatment of an inflammatory/autoimmune disease or disorder selected from a group consisting of hidradenitis suppurativa; ankylosing spondylitis; and primary sclerosing cholangitis; said method comprising administering to a subject in need of such prevention or treatment an effective amount of the crystalline form according to claim 1.

12. A method for the prevention or treatment of a disease or disorder selected from a group consisting of psoriasis, said method comprising administering to a subject in need of such prevention or treatment an effective amount of the crystalline form according to claim 1.

13. A method for the prevention or treatment of cancer, said method comprising administering to a subject in need of such prevention or treatment an effective amount of the crystalline form according to claim 1, wherein the crystalline form is used as a single therapeutic agent or in combination with one or more chemotherapy agents and/or radiotherapy and/or targeted therapy.

14. A method for the prevention or treatment of cancer, said method comprising administering to a subject in need of such prevention or treatment an effective amount of the crystalline form according to claim 1, wherein the crystalline form is used as a single therapeutic agent or in combination with one or more chemotherapy agents and/or radiotherapy and/or targeted therapy; wherein said cancer is selected from a group consisting of lymphoma; brain cancer; breast cancer; colorectal cancer; hepatocarcinomas; renal cell carcinoma; lung cancer; and gastric cancer.

15. A method for the prevention or treatment of cancer or an inflammatory/autoimmune disease or disorder, said method comprising administering to a subject in need of such prevention or treatment an effective amount of the crystalline form according to claim 1.

16. A pharmaceutical composition comprising the crystalline form according to claim 2, and at least one pharmaceutically acceptable carrier material.

17. A pharmaceutical composition comprising the crystalline form according to claim 3, and at least one pharmaceutically acceptable carrier material.

18. A pharmaceutical composition comprising the crystalline form according to claim 4, and at least one pharmaceutically acceptable carrier material.

19. A pharmaceutical composition comprising the crystalline form according to claim 5, and at least one pharmaceutically acceptable carrier material.

20. A process for preparing a pharmaceutical composition comprising 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol adipic acid salt, wherein said process comprises mixing a crystalline form of 2-(3-{5-[(R)-(1,3-dimethyl-azetidin-3-yl)-hydroxy-(4-isopropyl-phenyl)-methyl]-pyridin-3-yl}-[1,2,4]oxadiazol-5-yl)-propan-2-ol adipic acid salt characterized by the presence of peaks in the X-ray powder diffractogram at the following angles of refraction 2θ: 6.9°, 17.8° and 19.2°, and at least one pharmaceutically acceptable carrier material.
